(19) United States
(12) Patent Application Publication
(10) Pub. No.: US 2026/0146202 A1
(43) Pub. Date: May 28, 2026

(54) NON-IONIC SURFACTANT COMPOSITION FOR THE PREPARATION OF AQUEOUS EMULSIONS

(71) Applicant: ARKEMA FRANCE, Puteaux (FR)

(72) Inventor: Lise DEVES, Pierre-Benite Cedex (FR)

(73) Assignee: ARKEMA FRANCE, Puteaux (FR)

(21) Appl. No.: 19/121,507

(22) PCT Filed: Oct. 19, 2023
(86) PCT No.: PCT/FR2023/051635

§ 371 Date: Apr. 16, 2025

(30) Foreign Application Priority Data
Oct. 21, 2022 (FR) FR2210944

(51) Int. Cl. C10C 3/16

(52) U.S. Cl. CPC C10C 3/16

(57) ABSTRACT

The present invention relates to a composition comprising at least one non-ionic surfactant TA1 which is a fatty alcohol alkoxylate and at least one nonionic surfactant TA2 which is a phenol alkoxylate substituted with at least one hydrocarbon chain having from 10 to 60 carbon atoms. The present invention also relates to the use of said composition for preparing aqueous emulsions, in particular bitumen emulsions.

### BACKGROUND OF THE INVENTION

The present invention relates to compositions, in particular compositions based on surfactants which are useful in particular for the preparation of emulsions, more specifically for the preparation of bitumen emulsions, and even more specifically compositions based on nonionic surfactants, having good stability properties which are particularly suitable for the preparation of emulsions in various applications, in particular for the preparation of bitumen emulsions.

The market for surfactants for bitumen emulsions is constantly growing. These additives are of most particular importance for bitumen emulsions, insofar as they play an important role not only in the stability of bitumen emulsions but also in the robustness of the bituminous surfacing mixes prepared with said emulsions.

The use of surfactants of the nonionic type, notably in formulation, is well known to those skilled in the art and for the preparation of bitumen emulsions, nonionic emulsifiers are usually used with cationic co-emulsifiers. Nonionic emulsifiers are also often used, for example to compatibilize the lignin present in certain bitumen emulsion formulations.

U.S. Pat. No. 3,126,350 describes the manufacture of super-stabilized cationic bitumen emulsions using a chemically modified lignin. The chemical modifications impose additional operations and constraints, which may be a drawback as regards industrial preparation and the cost price of the resulting bitumen emulsions.

Other types of surfactant have been studied extensively, and for example U.S. Pat. No. 3,859,227 mentions slow-breaking cationic bituminous formulations comprising at least one alkoxylated alkylphenol. However, such alkoxylated alkylphenols are strictly controlled, these products nowadays being considered as potentially presenting risks to human health. Specifically, the ethoxylated alkylphenols used in bitumen emulsions are certainly recognized as being very good emulsifiers, but it has been shown that they degrade into alkylphenols, which are now recognized as endocrine disruptors.

The prior art provides even more examples of nonionic surfactants for use in bitumen emulsions. Thus, U.S. Pat. No. 3,366,500 claims an aggregate coating composition for road surfacing mix comprising a bitumen, an ethoxylated aromatic alcohol ether as hydrophilic nonionic surfactant, and a fatty acid alcohol ester as lipophilic nonionic surfactant. The examples mention compositions comprising ethoxylated nonylphenols and fatty acid esters having a long alkyl chain.

Patent FR2076630, for its part, discloses a bituminous binder in the form of an emulsion comprising a nonionic emulsifier of the long-chain alkyl ethoxylate type, this emulsifier in particular being a linear or branched long-chain fatty alcohol ethoxylate.

Patent application CN105013391 presents a method for preparing an ethoxylated cardanol containing 2 to 30 ethylene-oxy (EO) units. This compound is described as a surfactant which can replace ethoxylated nonylphenols for the preparation of bitumen emulsions having a high and controlled viscosity. However, this surfactant does not afford an emulsion that is sufficiently stable over time.

Patent application FR 2 782 724 also discloses bitumen emulsions of controlled viscosity, this time comprising two emulsifiers, the first promoting oil-in-water emulsification and the second promoting water-in-oil emulsification. This specific combination of surfactants leads to fast-breaking emulsions.

It emerges from the elements of the prior art that ethoxylated alkylphenols are known as emulsifiers or dispersants in many diverse and varied fields, for instance emulsion polymerization, bitumen emulsions, paints and coatings in general, to mention only their main uses. They allow emulsions or dispersions to be obtained which are characterized by their high stability. However, and as already indicated above, certain ethoxylated alkylphenols should no longer be used today because of their degradation products, which are recognized as endocrine disruptors, or are simply no longer used because their emulsion stabilizing properties are too weak.

The aim of the present invention consequently consists in finding novel emulsifiers or combinations of nonionic emulsifiers making it possible to obtain stable emulsions with a high degree of versatility to replace ethoxylated alkylphenols, notably those linked to their degradation products which are now considered toxic, in all its applications and more particularly for the preparation of bituminous compositions, and even more particularly for the preparation of bitumen emulsions. Other objects will emerge on reading the following description of the invention.

The inventors have now found that the abovementioned objectives are achievable, entirely or at least in part, by means of the composition according to the present invention. Specifically, the inventors have discovered a new combination of emulsifiers which allows stable emulsions to be obtained in many applications and more particularly for the preparation of bituminous compositions, and even more particularly for the preparation of bitumen emulsions.

### SUMMARY OF THE INVENTION

Thus, and according to a first aspect, the present invention relates to a composition comprising:
a) at least one nonionic surfactant TA₁ which is a fatty alcohol alkoxylate, and
b) at least one nonionic surfactant TA₂ which is a phenol alkoxylate substituted with at least one hydrocarbon-based chain including from 10 to 60 carbon atoms.

### DETAILED DESCRIPTION OF THE INVENTION

The nonionic surfactant TA₁ is an alkoxylated fatty alcohol. The term “fatty alcohol” means a primary, secondary or tertiary alcohol of formula R¹—OH, where R¹ is chosen from a linear or branched hydrocarbon-based radical including from 4 to 30 carbon atoms, preferably from 5 to 30 carbon atoms, more preferably from 6 to 30 carbon atoms, more generally from 6 to 20 carbon atoms, advantageously from 6 to 18 carbon atoms. The hydrocarbon-based radical R¹ may contain one or more unsaturations in the form of a double bond and/or a triple bond and/or a saturated or partially or totally unsaturated ring. The fatty alcohol of formula R¹—OH is a non-phenolic alcohol, i.e. the —OH group is not attached to an aromatic ring containing 6 carbon atoms. For the purposes of the invention, saturated hydrocarbon-based chains R¹ or chains including at least one double or triple bond are preferred; more preferably, the hydrocarbon-based chain R¹ is a linear or branched alkyl chain.

In addition, it should be understood that the term “fatty alcohol” also comprises mixtures of two or more primary, secondary or tertiary alcohols of formula R¹—OH, most particularly when the fatty alcohols are derived from renewable starting materials, for example from natural products.

According to a preferred embodiment of the invention, the alcohol of formula R¹—OH is a primary alcohol chosen from n-butanol, n-pentanol, n-hexanol, 2,2-dimethylbutanol, 2,3-dimethylbutanol, 3,3-dimethylbutanol, n-heptanol, n-octanol, 2-ethylhexanol, n-nonanol, n-decanol, isodecanol, n-undecanol, n-dodecanol and isotridecanol. However, this list is not exhaustive and other primary alcohols may also be mentioned.

According to another embodiment of the invention, the alcohol of formula R¹—OH is a secondary alcohol chosen from 2-butanol, 2-pentanol, 2-hexanol, 3,3-dimethyl-2-butanol, 2-heptanol, 3-heptanol, 4-methyl-2-pentanol, 2-octanol, 3-octanol, 4-octanol, 2-nonanol, 3-nonanol, 4-nonanol, 2-decanol, 3-decanol, 2-undecanol, 3-undecanol, 2-dodecanol and 3-dodecanol. However, this list is not exhaustive and other secondary alcohols may also be mentioned.

Among the alcohols of formula R¹—OH, alcohols chosen from 2-octanol, 2-ethylhexanol, isodecanol, isotridecanol and 4-methyl-2-pentanol are particularly preferred.

To form the nonionic surfactant TA₁, the fatty alcohol of formula R¹—OH described above is alkoxylated via any method well known to those skilled in the art. Specifically, it has been shown that the alkoxy units present in the fatty alcohol of formula R¹—OH participate in the formation of the desired emulsion. According to a preferred embodiment of the invention, the number of alkoxy units in the nonionic surfactant TA₁ is between 6 and 100, more preferably between 7 and 50, more preferentially between 8 and 50, better still between 8 and 30.

The alkoxy unit generally includes from 1 to 4 carbon atoms, more generally from 2 to 4 carbon atoms, and may optionally include one or more hydroxyl functions, more specifically optionally a hydroxyl function.

According to one embodiment of the invention, the alkoxy unit is chosen from ethylene-oxy, propylene-oxy, butylene-oxy and glycidyl-oxy units. The term “ethylene-oxy” means the —CH₂—CH₂—O unit, the term “propylene-oxy” means the —O—CH₂—CH(CH₃)— unit or the —CH₂—CH(CH₃)—O— unit, the term “butylene-oxy” means the —O—CH₂—C(C₂H5)H— unit, or the —CH₂—C(C₂H₅)H—O unit, and the term “glycidyl-oxy” means the —O—CH(CH₂OH)—CH₂— unit, the —CH(CH₂OH)—CH₂—O— unit, the —O—CH₂—CH(OH)—CH₂— unit, or the —CH₂—CH(OH)—CH₂—O-unit. Needless to say, the alkoxy units of the nonionic surfactant TA₁ may be identical or different, and when they are different, they may be distributed randomly, alternately, in blocks or in any combination of these distributions. In general, and in the usual manner, the alkoxylated chain of the nonionic surfactant TA₁ is terminated with a hydroxyl group (—OH).

For the purposes of the invention, and according to a preferred embodiment, the nonionic surfactant TA₁ includes identical alkoxy units, and more preferably the nonionic surfactant TA₁ includes ethoxy units. Examples of nonionic surfactants TA₁ are those sold under the names Neoliens®, Sensio® and Ensoline® by Arkema, Berol® by Nouryon, Lutensol®, Pluronic® or Plurafac® by BASF, Emulsogen® or Genapol® by Clariant, Mariipal® or Lialet® by Sasol, and Greenbentin® or Imbentin® by Kolb.

Among the preferred nonionic surfactants TA₁, nonlimiting examples that may be mentioned include 2-octanol bearing 15 ethoxy units (also noted as 2-octanol 15OE), 2-octanol bearing from 8 to 30 ethoxy units, 2-ethylhexanol bearing from 8 to 30 ethoxy units, 4-methyl-2-pentanol bearing from 8 to 30 ethoxy units, isodecanol bearing from 8 to 30 ethoxy units, and isotridecanol bearing from 8 to 30 ethoxy units.

As indicated previously, the composition according to the invention comprises, in addition to said at least one nonionic surfactant TA₁ explained above, at least one nonionic surfactant TA₂ which is a phenol alkoxylate substituted with at least one hydrocarbon-based chain R² including from 10 to 60 carbon atoms.

The hydrocarbon-based chain R², substituent of the phenolic group of the nonionic surfactant TA₂, is linear or branched and generally comprises from 10 to 60 carbon atoms, preferably from 12 to 50 carbon atoms, more preferably from 12 to 30 carbon atoms. The hydrocarbon-based chain R² may contain one or more unsaturations in the form of a double bond and/or a triple bond and/or a saturated or partially or totally unsaturated ring. For the purposes of the invention, hydrocarbon-based chains R² which are saturated or include at least one double or triple bond are preferred; more preferably, the hydrocarbon-based chain R² is a linear or branched hydrocarbon-based chain which is saturated or includes from one to three double bonds.

The phenolic group of the nonionic surfactant may also comprise one or more other substituents, for instance, as nonlimiting examples, one or more substituents chosen from an alkyl radical containing from 1 to 4 carbon atoms in a linear or branched chain, and a hydroxyl group (—OH).

According to a preferred embodiment of the invention, the phenol substituted with at least one hydrocarbon-based chain R² is chosen from phenolic compounds substituted with a hydrocarbon-based chain containing 15 carbon atoms, and optionally including one, two or three double bond(s), phenolic compounds substituted both with a second hydroxyl function and with a hydrocarbon-based chain containing 15 carbon atoms, and optionally including one, two or three double bond(s), phenolic compounds substituted with a second hydroxyl function, with a methyl group (—CH₃) and also with a hydrocarbon-based chain containing 15 carbon atoms, and optionally including one, two or three double bond(s). Among the compounds listed above, preference is given most particularly to the compounds known under the names cardanol, cardol and methylcardol, which are mixtures of phenols substituted with saturated and unsaturated (1, 2 and 3 double bonds) linear hydrocarbon-based chains containing 15 carbon atoms.

To form the nonionic surfactant TA₂, the phenol substituted with at least one hydrocarbon-based chain R² described above is alkoxylated, according to any method well known to those skilled in the art. The number of alkoxy units of the nonionic surfactant TA₂ is between 2 and 100, preferably between 4 and 100, more preferably between 10 and 100, even more preferably between 10 and 80, for example between 15 and 70.

The alkoxy unit of the nonionic surfactant TA₂ generally includes from 1 to 4 carbon atoms, more generally from 2 to 4 carbon atoms, and may optionally include one or more hydroxyl functions, more specifically optionally a hydroxyl function.

According to one embodiment of the invention, the alkoxy unit of the nonionic surfactant TA₂ is chosen from ethylene-oxy, propylene-oxy, butylene-oxy and glycidyl-oxy units, where ethylene-oxy, propylene-oxy, butylene-oxy and glycidyl-oxy are as defined previously. Needless to say, the alkoxy units of the nonionic surfactant TA₂ may be identical or different, and when they are different, they may be distributed randomly, alternately, in blocks or in any combination of these distributions.

For the purposes of the invention, and according to a preferred embodiment, the nonionic surfactant TA₂ includes identical alkoxy units, and more preferably the nonionic surfactant TA₂ includes ethoxy units. As for the nonionic surfactant TA₁, as a general rule and in the usual manner, the alkoxylated chain of the nonionic surfactant TA₂ is terminated with a hydroxyl group (—OH).

Among the preferred nonionic surfactants TA₂, nonlimiting examples that may be mentioned include cardanols bearing from 15 to 60 ethoxy units, for example cardanol bearing 30 ethoxy units (also noted as cardanol 30 OE).

The composition of the present invention can be readily prepared by simply mixing at least one nonionic surfactant TA₁ and at least one nonionic surfactant TA₂, according to any conventional mixing method well known to those skilled in the art. Alternatively, the composition of the invention may be prepared directly by mixing said at least one nonionic surfactant TA₁ and said at least one nonionic surfactant TA₂, at the time of use.

In the composition according to the present invention, the proportions of nonionic surfactant(s) TA₁ and of nonionic surfactant(s) TA₂ may vary within wide proportions, according to the desired effect and the use for which the composition is intended. In general, the TA₁/TA₂ weight ratio is between 1/99 and 99/1, preferably between 5/95 and 95/5, even more preferably between 10/90 and 90/10, better still between 15/85 and 85/15, and most usually between 20/80 and 80/20, for example 25/75 and 75/25, or even 40/60 and 60/40.

Particularly preferred examples of compositions according to the invention comprise, by way of nonlimiting example, mixtures of alkoxylated 2-octanol and alkoxylated cardanol, alkoxylated 2-ethylhexanol and alkoxylated cardanol, alkoxylated 4-methyl-2-pentanol and alkoxylated cardanol, alkoxylated isodecanol and alkoxylated cardanol, alkoxylated isotridecanol and alkoxylated cardanol. More specifically, nonlimiting examples that may be mentioned include mixtures of ethoxylated 2-octanol and ethoxylated cardanol, and more particularly mixtures of 2-octanol 15 OE and cardanol 30 OE, mixtures of 2-octanol 20 OE and cardanol 30 OE, mixtures of 2-octanol 15 OE and cardanol 60 OE, mixtures of 2-octanol 20 OE and cardanol 60 OE, and most particularly:
2-octanol 15 OE/cardanol 30 OE mixtures, in a 3/1 weight ratio,
2-octanol 15 OE/cardanol 30 OE mixtures, in a 1/1 weight ratio,
2-octanol 15 OE/cardanol 30 OE mixtures, in a 1/3 weight ratio,
2-octanol 15 OE/cardanol 60 OE mixtures, in a 3/1 weight ratio,
2-octanol 15 OE/cardanol 60 OE mixtures, in a 1/1 weight ratio,
2-octanol 15 OE/cardanol 60 OE mixtures, in a 1/3 weight ratio,
2-octanol 20 OE/cardanol 30 OE mixtures, in a 3/1 weight ratio,
2-octanol 20 OE/cardanol 30 OE mixtures, in a 1/1 weight ratio,
2-octanol 20 OE/cardanol 30 OE mixtures, in a 1/3 weight ratio,
2-octanol 20 OE/cardanol 60 OE mixtures, in a 3/1 weight ratio,
2-octanol 20 OE/cardanol 60 OE mixtures, in a 1/1 weight ratio, and
2-octanol 20 OE/cardanol 60 OE mixtures, in a 1/3 weight ratio.

The composition according to the present invention may also comprise one or more other surfactants chosen from anionic, cationic and amphoteric surfactants and zwitterions, such as, and by way of nonlimiting examples, surfactants chosen from amine surfactants, phosphate surfactants, surfactants with a quaternary amine function, surfactants with a sulfate function, surfactants with a sulfonate function, surfactants with a carboxylate function, betaines, and others.

Similarly, the composition according to the invention may comprise, if desired or if necessary, one or more additives and fillers well known to those skilled in the art and most often chosen from rheology modifiers, viscosity modifiers, pH modifiers, dyes, pigments, flavorings, preserving agents, natural or synthetic polymers and others, to mention only those most commonly used.

The composition according to the invention may advantageously be used to form emulsions, and in particular bitumen emulsions. To this end, the composition according to the invention may be supplemented with water and with acid so as to form a soap. The amount of water that may be present in the soap may vary within very wide proportions and may generally be between 0 and 99.99% by weight of water relative to the total weight of the soap, limits exclusive, preferably between 1% and 99% by weight of water relative to the total weight of the soap, limits inclusive.

According to a preferred embodiment, the soap as defined above is in the form of an “acidic” soap. For the purposes of the present invention, the term “acidic” means that the pH of the soap is less than 7, preferably less than 6; more preferably, it is between 0 and 7, preferably between 1 and 6, preferably between 1 and 4, for example 2. The pH value may be adjusted by adding a strong or weak organic or mineral acid.

Preferably, the pH value is adjusted, for example, by adding at least one acid chosen, for example, and in a nonlimiting manner, from phosphoric acids, hydrochloric acid, acetic acid, methanesulfonic acid and mixtures of two or more thereof in all proportions. The composition according to the present invention, and the soaps prepared from said composition, are generally in liquid form at ambient temperature, of homogeneous appearance, and free of precipitate.

As indicated previously, the composition according to the present invention, and the soaps prepared from said composition, may optionally comprise one or more additives commonly used in the field, among which mention may be made in a nonlimiting manner of rheological agents, colorants, stabilizers, surfactants, and also any other additive that is well known to those skilled in the art. According to a preferred aspect, the additives that may be present do not include a (hetero)aromatic nucleus; entirely preferably, the composition of the invention, and the soaps containing same, do not contain any alkoxylated nonylphenol.

It has been observed, entirely surprisingly, that, unlike the nonionic surfactants TA₁ and TA₂ taken separately, the composition of the present invention, which comprises at least one nonionic surfactant TA₁ and at least one nonionic surfactant TA₂, makes it possible to obtain stable emulsions which have good properties, in particular when used for emulsion manufacture, in terms of viscosity and particle size, inter alia. It may consequently be envisaged to use the composition of the present invention in all fields of application in which ethoxylated nonylphenols are generally used, which may degrade into nonylphenols, now listed as endocrine disruptors.

Examples of applications of the composition according to the present invention comprise, in a nonlimiting manner, the field of coatings, paints, fertilizers, bitumen, petroleum, detergents, cosmetics, emulsion polymerization, treatment of plant crops, biofuels, to mention only the main ones, and more particularly any possible use where it is necessary to have stable emulsions. It should be noted that the nonionic surfactants TA₁ and TA₂ are particularly suitable for use as surfactants for emulsions of the oil-in-water type.

Among the possible uses, the composition according to the present invention is of most particular interest for the preparation of bitumen emulsions. Thus, and according to a second aspect, the present invention relates to the use of a composition comprising at least one nonionic surfactant TA₁ and at least one nonionic surfactant TA₂, as defined above for the production of bitumen emulsions.

The invention thus relates to the use of at least two nonionic surfactants TA₁ and TA₂ as have just been defined for producing aqueous emulsions, and in particular bitumen emulsions. The emulsions prepared with the nonionic surfactants TA₁ and TA₂ as have just been defined, and which are free of ethoxylated nonylphenols, have, notably in terms of particle size distribution and stability, and irrespective of the field of application, properties which are completely equivalent to those observed with said ethoxylated nonylphenols, which are no longer desired for the reasons already mentioned previously. Thus, the present invention relates to the use of the composition described above as surfactants for the preparation of a bitumen emulsion.

It has thus been discovered, and this represents yet another aspect of the present invention, that bitumen emulsions prepared using the compositions according to the invention as defined above are stable and have good application properties as surfacing mixes.

According to one embodiment of the invention, the composition is used at low dosage, typically between 0.1% and 5%, preferably between 0.1% and 3%, more preferably between 0.5% and 2.0%, by weight of composition, limits inclusive, relative to the total weight of the bitumen emulsion.

The bitumen emulsions thus prepared comprise from 30% to 75% by weight of bitumen and preferably from 40% to 75% by weight of bitumen relative to the total weight of the emulsion, the remainder to 100% by weight being provided by water.

More specifically, the invention relates to a bitumen emulsion comprising:
from 30% to 75% by weight of bitumen and preferably from 40% to 75% by weight of bitumen, relative to the total weight of the emulsion,
from 0.1% to 5%, preferably from 0.1% to 3%, even more preferably from 0.5% to 2.0%, by weight of the composition defined previously, limits included, relative to the total weight of the bitumen emulsion, and
water, qs 100% by weight.

As already indicated above, the emulsion according to the present invention may optionally also comprise one or more other additives intended to modify its viscosity, to adjust its pH or the adhesiveness of the bitumen emulsion.

It has been observed, entirely surprisingly, that emulsions comprising the composition of surfactants TA₁ and TA₂ as defined previously have quite remarkable properties, and in particular in terms of stability overtime. These emulsions are also free of compounds which may generate degradation products now recognized as endocrine disruptors.

Another advantage of the emulsions of the invention comprising the composition of surfactants TA₁ and TA₂ lies in the fact that it is nonionic, which makes it possible to produce nonionic, cationic or anionic emulsions. Finally, a particularly suitable use for the composition of surfactants TA₁ and TA₂ is the preparation of “slow-breaking” bitumen emulsions.

The present invention also relates to a process for preparing a bitumen emulsion using at least one composition as described previously, said process comprising at least one step of mixing, preferably at high shear, at least one bitumen, at least one composition as defined previously and water. The order of addition (bitumen, composition, water) will be adapted as a function of the nature of the bitumen, of the composition and also of the knowledge of a person skilled in the art. The process also comprises an optional step of acidification of the medium so as to obtain a pH value as indicated previously.

The process is generally performed at a temperature that is sufficient to ensure good homogenization, as a function of the type of bitumen used. As a general rule, the emulsion is prepared at a temperature between ambient temperature and 160° C., preferably between 40° C. and 160° C., more preferably between 60° C. and 150° C. and more generally between 60° C. and 140° C., for example between 70° C. and 140° C. The bitumen emulsion preparation temperature may typically be adapted as a function of the penetrability of the bitumen used, as is well known to those skilled in the art.

According to a preferred aspect of the process for preparing the emulsion according to the present invention, a soap is prepared, at ambient temperature, or with gentle heating (for example from 30° C. to 80° C., preferably from 40° C. to 70° C., more preferably from 40° C. to 50° C.), by dispersing at least one composition as defined previously in water, and this soap is then acidified (generally to a pH of between 1.5 and 5 and preferably between 2 and 3.5) by adding at least one acid. The “acidic” soap obtained is then mixed at high shear with at least one bitumen at the temperatures indicated above.

The mixing at high shear may be performed using any apparatus known to those skilled in the art. By way of nonlimiting example, mention may be made of devices of colloid mill type, of which that of the Atomix® brand name is a representative.

Thus, and according to a preferred embodiment, the present invention relates to a process for preparing a bitumen emulsion as defined above, said process comprising:
at least one step of mixing, preferably at high shear, at least one bitumen, at least one composition as described previously and water,
an optional step of acidification of the medium so as to obtain a pH value of less than 7, preferably less than 6, more preferably between 0 and 7, preferably between 1 and 6, preferably between 1 and 4, for example 2,
said process being performed at a temperature between ambient temperature and 160° C., preferably between 40° C. and 160° C., more preferably between 60° C. and 150° C., more generally between 60° C. and 140° C., for example between 70° C. and 140° C.

The bitumen emulsion may optionally comprise one or more additives commonly used in the field, among which mention may be made in a nonlimiting manner of viscosity enhancers, natural or synthetic latices, thickeners, fluxing agents, plasticizers, and also any other additive for adjusting the properties of the emulsion, for instance the processing additives mentioned in patents EP 1 057 873, EP 2 035 504, EP 2 766 320 and EP 3 978 456.

The bitumens used in the invention are of any type known to a person skilled in the art, obtained from various sources, for example those of natural origin, those contained in natural bitumen deposits, natural asphalt deposits or bituminous sands. In the context of the present invention, the bitumens may also be bitumens obtained from refining crude oil (atmospheric and/or vacuum distillation of oil), these bitumens possibly being blown, visbroken and/or deasphalted bitumens.

The bitumens may be hard-grade or soft-grade bitumens. The various bitumens obtained by means of refining processes may be combined together to obtain the best technical compromise. The bitumens used may also be bitumens fluxed by addition of volatile solvents, of fluxing agents of petroleum origin, of carbochemical fluxing agents and/or of fluxing agents of plant origin.

Polymer-modified bitumens may also be used. Examples of polymers that may be mentioned, in a nonlimiting manner, include thermoplastic elastomers, for instance statistical or block copolymers of styrene and of butadiene, in linear or starburst form (SBR, SBS) or of styrene and isoprene (SIS), which are optionally crosslinked, copolymers of ethylene and of vinyl acetate, olefinic homopolymers and copolymers of ethylene (or propylene or butylene), polyisobutylenes, polybutadienes, polyisoprenes, poly(vinyl chloride)s, crumb rubbers or any polymer used for bitumen modification, and also mixtures thereof. An amount of polymer of from 2% to 10% by weight relative to the weight of bitumen is generally used.

Synthetic bitumens, also known as clear, pigmentable or colorable bitumens, may also be used. These bitumens contain little or no asphaltenes and can consequently be colored. These synthetic bitumens are based on petroleum resin and/or on indene-coumarone resin and on lubricating oil as described, for example, in patent EP 179510.

Advantageously, the bitumen is a bitumen with a penetrability, measured according to the standard EN1426, of between 10 and 300, preferably between 20 and 220, more preferentially between 70 and 220.

The invention also relates to surfacing mixes prepared with the abovementioned bitumen emulsions and aggregates. More particularly, the invention relates to surfacing mixes comprising at least one bitumen emulsion as described previously in the present description and aggregates.

The aggregates that may be used for the preparation of the surfacing mixes according to the present invention may be of any type known to those skilled in the art. Among the aggregates that may be used for the surfacing mixes of the present invention, mention may notably be made, in a nonlimiting manner, of aggregates of mineralogical nature, for example of eruptive nature, such as granites, porphyry stones and basalts, of metamorphic nature, such as schists and gneisses, and of sedimentary nature of siliceous type, such as silexes and quartzites, and of carbonated type, such as limestones and dolomites, but also surfacing mix (or recycled) aggregates, clinkers, crushed concretes, and the like, and also mixtures of such aggregates.

The surfacing mixes of the present invention may be prepared from at least one bitumen emulsion and aggregates according to any method known to those skilled in the art, for example by mixing the emulsion with the aggregates.

The emulsions of the present invention are thus particularly suitable for the preparation of surfacing mixes, for example semi-warm surfacing mixes, i.e. surfacing mixes in which the temperature of the aggregates during the phase of mixing with the bitumen emulsion is between ambient temperature and 100° C., for example also cold-laid surfacing mixes, among which mention may be made, in a nonlimiting manner, of gravel emulsions (structuring or reprofiling gravel emulsions), cold-laid bituminous concretes, dense, semi-dense or open storable surfacing mixes, and the like.

The emulsions of the present invention are also particularly suitable for making tack coats, for stabilizing grounds and for impregnation works, for instance “prime coat”, cold-in-place recycling (or CIPR), full depth reclamation (or FDR), the preparation of coatings, optionally in combination with one or more other additives and/or fillers, for instance clays, limestone powder or cement, in order to prepare sealing coatings and the like.

The use according to the present invention notably allows the replacement of ethoxylated nonylphenols, in bitumen emulsions, which are recognized as being very good emulsifiers but which degrade into nonylphenols, which are now listed as endocrine disruptors.

The examples that follow are intended to illustrate the present invention, without however limiting the scope thereof, which is defined by the appended claims.

### EXAMPLES

The examples that follow allow the efficacy of the composition according to the invention to be evaluated. In particular, these examples show that the composition according to the invention provides better results in comparison with surfactants taken separately and relative to a reference. As indicated later, the stability of bitumen emulsions can in particular be measured by cement stability tests (NF EN 12848) and by measurement of the breaking value (application of NF EN 13075-1 for a cationic, nonionic or anionic emulsion).

### Description of the Test Methods

The sedimentation tendency after 7 days of storage is assessed according to the standard NF EN 12847 (July 2009), “Determination of settling tendency of bitumen emulsions”. The emulsions are placed in a 100 mL graduated cylinder. On conclusion of 7 days storage at room temperature without agitation, the water content is determined on the upper 10 mL and the lower 10 mL. The emulsion is considered as stable if the difference in binder content between the two fractions is less than 5%.

The breaking value is measured according to the standard NF EN 13075-1 of December 2016, “Bitumens and bituminous binders—Determination of breaking behaviour—Part 1: Determination of the breaking value of cationic bitumen emulsions, mineral fines method” with a Sikaisol filler. The test consists in gradually adding filler to a bitumen emulsion while simultaneously stirring it. Breakage is evaluated visually from the moment at which the emulsion solidifies.

The homogeneity by screening is evaluated according to the standard NF EN 1429 (August 2013) “Bitumens and bituminous binders—Determination of the residue on sieving of bitumen emulsions and determination of storage stability by sieving”. The emulsion is passed through a 500 μm mesh size sieve. The sieve rejection is the emulsion residue remaining on the sieve on conclusion of this operation.

### Example 1: Preparation of Bitumen Emulsions

Bitumen emulsions are prepared with two nonionic surfactants separately or in combination and with ethoxylated nonylphenol. Unless otherwise indicated, the measurements are expressed in kilograms per tonne (kg/to). The contents of the emulsions are detailed in Table 1 below:

TABLE 1

| | R1 | R2 | R3 | E1 | E2 | E3 |
|---|---|---|---|---|---|---|
| | Bitumen phase: 650 kg/to | | | | | |
| EXXON 70/100 | 650 | 650 | 650 | 650 | 650 | 650 |
| bitumen | | | | | | |
| | Aqueous phase: 350 kg/to | | | | | |
| Cardanol 30OE | 10 | — | — | 1.75 | 3.5 | 5.25 |
| 2-Octanol 15 OE | — | 7 | — | 5.25 | 3.5 | 1.75 |
| Nonylphenol 30 OE | — | — | 7 | — | — | — |
| Hydrochloric acid | 0.7 | 0.7 | 0.7 | 0.8 | 0.8 | 0.7 |
| Water | 339.3 | 342.3 | 342.3 | 342.2 | 342.2 | 342.3 |

Emulsion R1 is a reference emulsion containing only a nonionic surfactant of alkoxylated cardanol type, cardanol with 30 ethoxy units. Emulsion R2 is a reference emulsion containing only an ethoxylated branched fatty alcohol type nonionic surfactant, 2-octanol with 15 ethoxy units. Emulsion R3 is a reference emulsion containing ethoxylated nonylphenol. Emulsions E1, E2 and E3 are emulsions according to the invention containing mixtures of 2-octanol 15 OE and cardanol 30 OE in various proportions.

The emulsions are prepared according to an identical protocol using a gradual enrichment laboratory mill (sold by Herbert Rink Elektromaschinenbau GmbH). The properties of the emulsions obtained are shown in Table 2 below:

TABLE 2

| | R1 | R2 | R3 | E1 | E2 | E3 |
|---|---|---|---|---|---|---|
| pH of the | 2 | 2 | 2 | 2 | 2 | 2 |
| aqueous phase | | | | | | |
| (NF EN 12850) | | | | | | |
| Homogeneity | 3.89 | 7.57 | 0.21 | 0.09 | 0.03 | 0.21 |
| by sieving (% | | | | | | |
| of rejects at | | | | | | |
| 500 μm) | | | | | | |
| (NF EN 1429) | | | | | | |
| Breaking value | 164 | 38 | 171 | 193 | 194 | 180 |
| (NF EN 13075-1) | | | | | | |
| Settling tendency | 21.7 | * | 3.1 | 5.7 | 3.3 | 5.0 |
| after 7 days of | | | | | | |
| storage | | | | | | |
| (NF EN 12847) | | | | | | |

* not performable, start of breakage

This example shows a very marked improvement in the stability of the emulsion (higher IREC and improved storage stability) using a composition including two nonionic surfactants according to the invention, relative to emulsions prepared with each of the nonionic surfactants separately.

It is also observed that the properties of the emulsions according to the invention are very close to those of the emulsion prepared with an ethoxylated nonylphenol, thus demonstrating that the composition according to the invention can entirely replace ethoxylated nonylphenol for the preparation of emulsions.

### Example 2: Compatibility with Cationic Surfactants

Cationic bitumen emulsions of the spreading type are prepared. Unless otherwise indicated, the measurements are expressed in kilograms per tonne (kg/to). The contents of the emulsions are detailed in Table 3 below:

TABLE 3

| | |
|---|---|
| R4 | E4 |

Bitumen phase: 570 kg/to

| | | |
|---|---|---|
| 70/100 PETROINEOS bitumen | 570 | 570 |

Aqueous phase: 430 kg/to

| | | |
|---|---|---|
| Cardanol 30 OE | — | 2.5 |
| 2-Octanol 15 OE | — | 2.5 |
| Nonylphenol 30 OE | 5 | — |
| Quaternary ammonium cationic | 0.8 | 0.8 |
| surfactant* | | |
| Hydrochloric acid | 0.7 | 0.8 |
| Water | 423.5 | 423.4 |

*Stabiram ® MS6 (Arkema)

Emulsion R4 is a reference emulsion containing ethoxylated nonylphenol and a quaternary ammonium cationic co-surfactant. Emulsion E4 is an emulsion according to the invention comprising a nonionic surfactant TA₁, a nonionic surfactant TA₂, and the same quaternary ammonium cationic co-surfactant.

The emulsions are prepared according to an identical protocol with an automatic Emulbitume laboratory mill comprising an Atomix® brand mixer. The properties of the emulsions obtained are shown in Table 4 below:

TABLE 4

| | R4 | E4 |
|---|---|---|
| pH of the aqueous phase | 2 | 2 |
| (NF EN 12850) | | |
| Homogeneity by sieving (% of | 0.38 | 0.13 |
| rejects at 500 μm) (NF EN 1429) | | |
| Breaking value | 202 | 193 |
| (NF EN 13075-1) | | |
| Settling tendency after 7 days of | 8.8 | 9.8 |
| storage (NF EN 12847) | | |

This example shows that the composition according to the invention, in combination with a cationic surfactant, affords an emulsion with similar or even better properties, notably in terms of stability, compared with the properties obtained with an emulsion based on ethoxylated nonylphenol and the same cationic surfactant. It is thus also demonstrated here that the composition according to the invention can entirely replace ethoxylated nonylphenol for the preparation of emulsions.

### Example 3: Effect of the Number of Alkoxy Units

Bitumen emulsions are prepared with mixtures of nonionic surfactants, the ethoxylated fatty alcohol present having a number of ethoxylates of 8 (E5 and E6) and 4 (Comparative example). The emulsions are prepared according to a protocol identical to that described in Examples 1 and 2 of the patent using a gradual enrichment laboratory mill (sold by Herbert Rink Elektromaschinenbau GmbH).

Unless otherwise indicated, the measurements are expressed in kilograms per tonne (kg/to). The contents of the emulsions are detailed in Table 5 below:

TABLE 5

| | | | Comparative |
|---|---|---|---|
| | E5 | E6 | example |
| | Bitumen phase: | Bitumen phase: | Bitumen phase: 650 |
| | 650 kg/to | 650 kg/to | kg/to |
| EXXON 70/100 | 650 | 650 | 650 |
| bitumen | | | |
| | Aqueous phase: | Aqueous phase: | Aqueous phase: 350 |
| | 350 kg/to | 350 kg/to | kg/to |
| Cardanol 30 OE | 3.5 | | 3.5 |
| Cardanol 25 OE | | 3.5 | |
| 2-Octanol 8 OE | 3.5 | 3.5 | |
| 2-Octanol 4 OE | | | 3.5 |
| Hydrochloric acid | 0.8 | 0.8 | 0.8 |
| Water | 342.2 | 342.2 | 342.2 |

The properties of the emulsions are shown in Table 6 below:

TABLE 6

| | | | Comparative |
|---|---|---|---|
| | E5 | E6 | example |
| pH of the aqueous phase | 2 | 2 | — |
| (NF EN 12850) | | | |
| Homogeneity by sieving | 0.11 | 0.15 | — |
| (% of rejects at 500 | | | |
| μm) (NF EN 1429) | | | |
| Breaking value (NF EN 13075-1) | 185 | 183 | — |
| Settling tendency after 7 days | 4.2 | 3.9 | — |
| of storage (NF EN 12847) | | | |

The features of the emulsions obtained with E5 and E6, both comprising 2-octanol containing 8 OE, are entirely compliant in terms of homogeneity, breaking value and sedimentation tendency. On the other hand, for the comparative example, including 2-octanol containing 4 OE, the emulsion does not form. This example shows the very strong dependence of the emulsifying capacity of the mixture on the number of alkoxy units in the fatty alcohol used.

1. A composition comprising:
a) at least one nonionic surfactant TA₁ which is a fatty alcohol alkoxylate, and
b) at least one nonionic surfactant TA₂ which is a phenol alkoxylate substituted with at least one hydrocarbon-based chain including from 10 to 60 carbon atoms.

2. The composition as claimed in claim 1, in which the nonionic surfactant TA₁ is an alkoxylated fatty alcohol, the fatty alcohol being a primary, secondary or tertiary alcohol of formula R₁—OH, where R¹ is chosen from a linear or branched hydrocarbon-based radical including from 4 to 30 carbon atoms.

3. The composition as claimed in claim 1, in which the nonionic surfactant TA₁ is an alkoxylated fatty alcohol, the fatty alcohol being an alcohol chosen from n-butanol, n-pentanol, n-hexanol, 2,2-dimethylbutanol, 2,3-dimethylbutanol, 3,3-dimethylbutanol, n-heptanol, n-octanol, 2-ethylhexanol, n-nonanol, n-decanol, isodecanol, n-undecanol, n-dodecanol, isotridecanol, 2-butanol, 2-pentanol, 2-hexanol, 3,3-dimethyl-2-butanol, 2-heptanol, 3-heptanol, 4-methyl-2-pentanol, 2-octanol, 3-octanol, 4-octanol, 2-nonanol, 3-nonanol, 4-nonanol, 2-decanol, 3-decanol, 2-undecanol, 3-undecanol, 2-dodecanol and 3-dodecanol.

4. The composition as claimed in claim 1, in which the nonionic surfactant TA₁ is an alkoxylated fatty alcohol, the number of alkoxy units in the nonionic surfactant TA₁ being between 6 and 100.

5. The composition as claimed in claim 1, in which the nonionic surfactant TA₁ is chosen from 2-octanol bearing 15 ethoxy units, 2-octanol bearing from 8 to 30 ethoxy units, 2-ethylhexanol bearing from 8 to 30 ethoxy units, 4-methyl-2-pentanol bearing from 8 to 30 ethoxy units, isodecanol bearing from 8 to 30 ethoxy units, and isotridecanol bearing from 8 to 30 ethoxy units.

6. The composition as claimed in claim 1, in which the nonionic surfactant TA₂ is a phenol alkoxylate substituted with at least one linear or branched hydrocarbon-based chain R² including from 10 to 60 carbon atoms.

7. The composition as claimed in claim 1, in which the nonionic surfactant TA₂ is a substituted phenol alkoxylate chosen from cardanol, cardol and methylcardol.

8. The composition as claimed in claim 1, in which the nonionic surfactant TA₂ is a phenol alkoxylate substituted with at least one hydrocarbon-based chain comprising from 10 to 60 carbon atoms, and comprising a number of alkoxy units of between 2 and 100.

9. The composition as claimed in claim 1, in which the nonionic surfactant TA₂ is chosen from cardanols bearing from 15 to 60 ethoxy units.

10. The composition as claimed in claim 1, in which the TA₁/TA₂ weight ratio is between 1/99 and 99/1.

11. The composition as claimed in claim 1, comprising mixtures chosen from:
2-octanol 15 OE/cardanol 30 OE mixtures, in a 3/1 weight ratio,
2-octanol 15 OE/cardanol 30 OE mixtures, in a 1/1 weight ratio,
2-octanol 15 OE/cardanol 30 OE mixtures, in a 1/3 weight ratio,
2-octanol 15 OE/cardanol 60 OE mixtures, in a 3/1 weight ratio,
2-octanol 15 OE/cardanol 60 OE mixtures, in a 1/1 weight ratio,
2-octanol 15 OE/cardanol 60 OE mixtures, in a 1/3 weight ratio,
2-octanol 20 OE/cardanol 30 OE mixtures, in a 3/1 weight ratio,
2-octanol 20 OE/cardanol 30 OE mixtures, in a 1/1 weight ratio,
2-octanol 20 OE/cardanol 30 OE mixtures, in a 1/3 weight ratio,
2-octanol 20 OE/cardanol 60 OE mixtures, in a 3/1 weight ratio,
2-octanol 20 OE/cardanol 60 OE mixtures, in a 1/1 weight ratio, and
2-octanol 20 OE/cardanol 60 OE mixtures, in a 1/3 weight ratio.

12. The composition as claimed in claim 1, also comprising one or more other surfactants chosen from anionic, cationic and amphoteric surfactants and zwitterions.

13. Use of a composition as claimed in claim 1, for making aqueous emulsions.

14. The use of the composition as claimed in claim 13, as a surfactant for the preparation of a bitumen emulsion.

15. A bitumen emulsion comprising:
from 30% to 75% by weight of bitumen, relative to the total weight of the emulsion,
from 0.1% to 5%, by weight of the composition as claimed in claim 1, limits included, relative to the total weight of the bitumen emulsion, and
water, qs 100% by weight.
